(19) United States
(12) Patent Application Publication
(10) Pub. No.: US 2004/0137573 A1
(43) Pub. Date: Jul. 15, 2004

(54) Human vaniloid receptor-like proteins

(76) Inventors: Katsuhiro Shinjo, Chita-gun (JP); Hikaru Yabuuchi, Yokohama (JP)

Correspondence Address: Mehdi Ganjeizadeh, Warner-Lambert Company, 2800 Plymouth Road, Ann Arbor, MI, 48105, US

(21) Appl. No.: 10/761,065

(22) Filed: Jan. 20, 2004

Related U.S. Application Data
(63) Continuation of application No. 09/870,090, filed on May 30, 2001, now abandoned.
(60) Provisional application No. 60/208,156, filed on May 31, 2000.

(51) Int. Cl. C07K 14/705; C07H 21/04

(52) U.S. Cl. 435/69.1; 435/320.1; 435/325; 530/350; 536/23.5

(57) ABSTRACT

This invention relates to human vanilloid receptor-like protein 2 (VRL-2) polypeptides, polynucleotides encoding such polypeptides, polynucleotide probes or primers, expression vectors and host cells comprising such DNA molecules. This invention further relates to a process for producing the polypeptides; an antibody immunospecific for the polypeptide; a diagnostic kit for diagnosing the VRL-2 receptor related disease; a method for screening to identify modulators which modulate the polypeptides; modulators identified by the screening method; a pharmaceutical composition for treatment of conditions associated with biological function of the polypeptides; and a non-human transgenic animal model for vanilloid receptor-like gene. The polypeptides and the DNA molecules of the present invention can be used to identify agonists, antagonists or the like. These agonists and antagonists are useful for treatment of diseases such as pain, nociceptive pain, chronic pain, neuropathic pain, postoperative pain, cancer pain, rheumatoid arthritic pain, osteoarthritis, diabetic neuropathies, neuralgia, neuropathies, algesia, nerve injury, muscle-skeletal pain, low back pain, neurodegeneration, stroke, inflammatory disorders, athma, allergy, urogenital disorders, incontinence, hypertension, hypotension, perivasular disease and the like.

This application is a continuation of U.S. application Ser. 09/870,090, filed May 30, 2001 which application claims the priority of U.S. Provisional application 60/208,156 filed May 31, 2000; the entire contents of each of which applications are herby incorporated herein by reference.

### TECHNICAL FIELD

This invention relates to human vanilloid receptor-like protein 2 (VRL-2) polypeptides, polynucleotides encoding such polypeptides, polynucleotide probes or primers, expression vectors and host cells comprising such DNA molecules. This invention further relates to a process for producing the polypeptides; an antibody immunospecific for the polypeptide; a diagnostic kit for diagnosing the VRL-2 receptor related disease; a method for screening to identify modulators which modulate the polypeptides; modulators obtainable by the screening method; a pharmaceutical composition for treatment of conditions associated with biological function of the polypeptides; and a non-human transgenic animal model for vanilloid receptor-like gene.

### BACKGROUND ART

The analgesic properties of capsaicin and capsaicinoides are known for their uses in the treatment of a variety of disorders such as pain, chronic pain, neuropathic pain, postoperative pain, rheumatoid arthritic pain, neuralgia, neuropathies, algesia, nerve injury, ischaemia, neurodegeneration stroke incontinence and inflammatory disorders (e.g., Campbell et al. “Clinical Applications of Capsaicin and Its Analogues” in Capsaicin in the Study of Pain, Academic Press pgs. 255-272 (1993)).

Capsaicin receptors are believed to be members of the ion channel family of polypeptides. These receptors are believed to be associated with the mechanism of action of capsaicin (a vanilloid compound). Capsaicin elicits a senstation of burning pain by selectively activating sensory neurons that convey information about noxious stimuli to the central nervous system (e.g., Caterina, M. J. eta al., “The Capsaicin Receptor: A Heat Activated Ion Channel In the Pain Pathway”, Nature 389, 816-824 (1997) and Caterina, M. J. et al., “A Capsaicin-Receptor Homologue with A High Threshold For Noxious Heat”, Nature 398, 436-441 (1999)). The channels are permeable to cations and exhibit a notable preferance for divalent cations, particularly calcium ions. The level of calcium ion permeability exceeds that observed for most non-selective cation channels and is similar to values observed for NMDA-type glutamate receptors and alpha-7 nicotinic acetylcholine receptors.

Some polynucleotides encoding vanilloid receptors or vanilloid-like receptors have been cloned and identified (Caterina, M. J. eta al., Nature 389, 816-824 (1997) and Caterina, M. J. et al., Nature 398, 436-441 (1999)). PCT Application International Publication No. WO 99/37675 discloses vanilloid receptor polypeptides and vanilloid receptor-related polypeptides, specifically the capsaicin receptor subtypes VR1 and VR2, and the encoding polynucleotide sequences. PCT Application International Publication NO. WO 99/37765 discloses VANILREP2 polypeptides and polynucleotides, recombinant materials and methods for their production.

If other subtypes of the capsaicin receptors were found and identified, it would be beneficial, for example, to use such subtypes to find novel ligands with respect to the capsaicin receptors.

### BRIEF DISCLOSURE OF THE INVENTION

The present invention provides a polypeptide selected from:

(a) a polypeptide comprising an amino acid sequence of SEQ ID NO:2 or SEQ ID NO:4, or variants thereof; and

(b) a polypeptide having the deduced amino acid sequence translated from the polynucleotide sequence SEQ ID NO:1 or SEQ ID NO:3, or variants thereof.

Such polypeptides have been identified as novel human vanilloid receptor-like protein 2 polypeptides. As used herein, the novel human vanilloid receptor-like protein 2 is referred to as “VRL-2” or “VRL-2 protein”. As used herein, these polypetides can be referred to as “VRL-2 polypeptides” or “VRL-2 receptor polypeptides”.

The present invention also provides an isolated and/or purified polynucleotide selected from:

(a) a polynucleotide encoding the polypeptide comprising an amino acid sequence of SEQ ID NO:2 or SEQ ID NO:4;

(b) a polynucleotide having a nucleotide sequence of the 85th to 2700th nucleotides in the nucleotide sequence of SEQ ID NO:1, or a nucleotide sequence of the 43rd to 1851st nucleotides in the nucleotide sequence of SEQ ID NO:3 or variants thereof;

(c) a polynucleotide comprising a nucleotide sequence that has at least 70% identity to the polynucleotide of (a) or (b);

(d) a complement to the polynucleotide of any one of (a) to (c);

(e) a polynucleotide comprising a nucleotide sequence which is capable of hybridising to the polynucleotide of any one of (a) to (d); and

(f) a polynucleotide fragment of the polynucleotide of any one of (a) to (e).

The present invention also provides a polynucleotide probe or primer comprising at least 15 contiguous nucleotides of the polynucleotide described above, or the complement thereof.

Further, the present invention provides an expression vector for the expression of the VRL-2 protein in a recombinant host cell wherein said expression vector contains the above DNA molecule, and a host cell which expresses a recombinant VRL-2 protein wherein said host cell contains the expression vector. Also provided is a process for producing the VRL-2 receptor polypeptides, which comprises culturing the host cell under conditions sufficient for the production of said polypeptide and recovering the polypeptide from the culture medium.

The VRL-2 receptor polypeptides, the VRL-2 receptor-encoding DNA, the expression vectors and recombinant host cells expressing recombinant VRL-2 are useful in the identification of modulators which modulate the function of the VRL-2 receptor. Thus, the present invention also provides a method for screening to identify modulators which modulate (e.g., bind) the VRL-2 receptor polypeptide, which comprises contacting the polypeptide with a test compound or sample, and detecting an activity of the compound or sample to modulate the function of the VRL-2 receptor polypeptide.

The present invention further provides a VRL-2 receptor modulator (e.g., agonist and antagonist) obtainable by the above screening method. These VRL-2 receptor modulators are useful for the treatment of diseases or conditions associated with the function of the VRL-2 receptor, or inappropriate VRL-2 receptor activity. Such diseases include pain, nociceptive pain, chronic pain, neuropathic pain, postoperative pain, cancer pain, rheumatoid arthritic pain, osteoarthritis, diabetic neuropathies, neuralgia, neuropathies, algesia, nerve injury, muscle-skeletal pain, low back pain, neurodegeneration, stroke, inflammatory disorders, athma, allergy, urogenital disorders, incontinence, hypertension, hypotension, perivasular disease and the like.

Thus, the VRL-2 receptor modulators are useful in the treatment of such diseases. In addition, the VRL-2 receptor polypeptides and the VRL-2 receptor-encoding polynucleotides are also useful in diagnostic assays for detecting the VRL-2 receptor-related diseases.

The present invention further provides a diagnostic kit for diagnosing a disease or a susceptibility to a disease associated with biological function of a human vanilloid receptor-like protein, which comprises the above polynucleotides or an antibody to the VRL-2 receptor polypeptide.

In addition, the present invention provides an antibody immunospecific for the VRL-2 receptor polypeptides, and a non-human transgenic animal model for VRL-2 receptor gene function wherein the transgenic animal has an alteration in VRL-2 receptor function relative to a normal animal of the same species.

### BRIEF DESCRIPTION OF FIGURES

FIGS. 1(A) and 1(B) show Ca²⁺ increase in hVRL-2 transfected CHO-K1 cells in response to a reduction in extracellular osmolarity.

FIGS. 2(A) and 2(B) show Ca²⁺ increase in hVRL-2 transfected HEK293 cells in response to a reduction in extracellular osmolarity.

### DETAILED DESCRIPTION OF THE INVENTION

As used herein, the term “nucleotide sequence” refers to an oligonucleotide sequence or polynucleotide sequence, and variants, homologues, fragments and derivatives thereof (such as portions thereof). The nucleotide sequence may be DNA or RNA of genomic or synthetic or recombinant origin, which may be double-stranded or single-stranded whether representing the sense or antisense strand. Preferably, the term “nucleotide sequence” means DNA. More preferably, the term “nucleotide sequence” means DNA prepared by use of recombinant DNA techniques (i.e. recombinant DNA).

As used herein “amino acid sequence” refers to peptide or protein sequences or portions thereof.

As used herein, the terms “isolated” and “purified” refer to molecules, either nucleic or amino acid sequences, that are removed from their natural environment and isolated or separated from at least one other component with which they are naturally associated.

The terms “variant” in relation to the amino acid sequence for the preferred polypeptide of the present invention include any substitution of, variation of, modification of, replacement of, deletion of or addition of one or more amino acids from or to the sequence providing the resultant polypeptide has VRL-2 activity.

The terms “variant” or “fragment” in relation to the nucleotide sequence coding for the preferred polypeptide of the present invention include any substitution of, variation of, modification of, replacement of, deletion of or addition of one or more nucleic acids from or to the sequence providing the resultant nucleotide sequence codes for or is capable of coding for a polypeptide having VRL-2 activity. Relative sequence homology (i.e. sequence identity) can be determined by commercially available computer programs that can calculate % homology between two or more sequences. A typical example of such a computer program is CLUSTAL.

The term “variant” encompasses sequences that are complementary to sequences that are capable of hybridising under stringent conditions (e.g. 65° C. and 0.1×SSC {1×SSC=0.15 M NaCl, 0.015 M Na₃ citrate pH 7.0}) to the nucleotide sequences presented herein.

The term “expression vector” means a construct capable of in vivo or in vitro expression.

As used herein, the term “antibodies” include polyclonal and monoclonal antibodies, chimeric, single chain and humanized antibodies. The term “immunospecific” means that the antibodies have substantially greater affinity for the polypeptides of the invention than their affinity for other related polypeptides.

The present invention will be further described bellow.

The present invention is based on the identification of a polynucleotide sequence encoding a novel human vanilloid receptor-like protein 2 (VRL-2) polypeptide. Such a polynucleotide has been isolated and purified, and the nucleotide sequence has been determined.

VRL-2 Preceptor Polypeptides

In a first aspect, the present invention provides a polypeptide selected from:

(a) a polypeptide comprising an amino acid sequence of SEQ ID NO:2 or SEQ ID NO:4, or variants thereof; and

(b) a polypeptide having the deduced amino acid sequence translated from the polynucleotide sequence SEQ ID NO:1 or SEQ ID NO:3, or variants thereof.

The VRL-2 receptor polypeptides of the present invention are believed to be members of the ion channel family of polypeptides. The channels are permeable to cations and exhibit a notable preferance for divalent cations, particularly calcium ions. The permeability of cations and the signal transmission ability of the VRL-2 receptors are affected by a ligand which modulates (e.g., binds, inhibits and activates) the function of the VRL-2 receptor polypeptides. These properties are hereinafter referred to as “VRL-2 receptor activities”.

Preferably, the polypeptide or variants comprises an amino acid sequence that has at least 75% (more preferably at least 85%, even more preferably at least 95%) identity to the an amino acid sequence of SEQ ID NO:2 or SEQ ID NO:4. The variants preferably show at least one of the above-mentioned VRL-2 activities. More specifically, the polypeptides may vary from the referents by conservative amino acid substitutions, whereby a residue is substituted by another with the VRL-2 activity. Such substitutions may be made among Ala, Val, Leu and lie; among Ser and Thr; among the acidic residues Asp and Glu; among Asn and Gin; and among the basic residues Lys and Arg; or aromatic residues Phe and Tyr. Thus, this invention includes variants of the polypeptides where 1 to 10, more preferably 1 to 5, further preferably 1 to 3, further preferably 1 to 2 or most preferably 1 amino acid(s) are substituted, deleted, or added in any combination in the amino acid sequence of SEQ ID NO:2 or NO:4.

The polypeptides of the present invention may be in the form of the “mature” protein or may be a part of a larger protein such as a precursor or a fusion protein. It is often advantageous to include an additional amino acid sequence which contains secretory or leader sequences, prosequences, sequences which aid in purification such as multiple histidine residues, or an additional sequence for stability during recombinant production.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art. The VRL-2 receptor polypeptides of this invention can be prepared using DNA molecules encoding the amino acid sequence of the polypeptides.

Polynucleotides/DNA Molecules

In a further aspect, the present invention relates to an isolated and/or purified polynucleotide selected from:

(a) a polynucleotide encoding the polypeptide comprising an amino acid sequence of SEQ ID NO:2 or SEQ ID NO:4;

(b) a polynucleotide having a nucleotide sequence of the 85th to 2700th nucleotides in the nucleotide sequence of SEQ ID NO:1, or a nucleotide sequence of the 43rd to 1851st nucleotides in the nucleotide sequence of SEQ ID NO:3 or variants thereof;

(c) a polynucleotide comprising a nucleotide sequence that has at least 70% identity to the polynucleotide of (a) or (b);

(d) a complement to the polynucleotide of any one of (a) to (c);

(e) a polynucleotide comprising a nucleotide sequence which is capable of hybridising to the polynucleotide of any one of (a) to (d); and

(f) a polynucleotide fragment of the polynucleotide of any one of (a) to (e).

Preferably, the polynucleotide comprises a nucleotide sequence that has at least 75% (more preferably at least 85%, yet more preferably at least 95%) identity to the nucleotide sequence of the above polynucleotide (b).

The nucleotide sequences of SEQ ID NO: 1 and SEQ ID NO:3 show homology with rat vanilloid receptor 1 (VR1) (M. J. Caterina et al., Nature 389: 816-824, 1997), rat/human vanilloid receptor like protein 1 (VRL-1) (M. J. Caterina et al.,_Nature 398: 436-441, 1999), rat stretch-activated channel (SIC) (J. Biol. Chem. 274: 6330-6335, 1999) and mouse growth-factor-regulated channel (GRC) (Nature Cell Biol. 1: 165-170). The nucleotide sequences of SEQ ID NO:1 and SEQ ID NO:3 were found to be full length of cDNA encoding the VRL-2a and VRL-2b polypeptides, respectively. Similarities of human VRL-2a with rat VR1, rat VRL-1, human VRL-1, rat SIC and mouse GRC in amino acid sequence were 51%, 46%, 47%, 59% and 47%, respectively. Similarities of human VRL-2b with rat VR1, rat VRL-1, human VRL-1, rat SIC and mouse GRC in amino acid sequence were 50%, 45%, 46%, 59% and 45%, respectively. VR1 is heat, proton and capsaicin-activated cation channel. Rat VRL-1 is heat-activated cation channel. Rat SIC is mechano-sensitive cation channel. Mouse GRC is IGF-1-regulated cation channel. Accordingly, both human VRL-2a and VRL-2b are predicted to be cation channels activated by stumuli such as heat, proton, chemicals, mechano-stimulation of membrane, growth factor.

The polynucleotides (DNA molecules) of the present invention may be obtained, using standard cloning and screening techniques, from a cDNA library derived from mRNA in cells of human placenta, brain, heart, melanocytse, liver, spleen, foreskin, lung, parathyroid, tonsil, whole embryo, Jurkat T-cells, B-cells and testes (for example Sambrook et al., Molecular Cloning: A Laboratory Manual, 2nd Ed., Cold Spring Harbor Laboratory Press, Cold Spring Harbor, N.Y. (1989)). The DNA molecules of the invention can also be obtained from natural sources such as genomic DNA libraries or can be synthesized using well known and commercially available techniques.

Other cells and cell lines may also be suitable for use to isolate VRL-2 cDNA. Selection of suitable cells may be done by screening for VRL-2 on cell surfaces. Methods for detecting VRL-2 activity are well known in the art and measure the binding of radiolabelled ligand specific for the receptor. Cells which possess VRL-2 activity in this assay may be suitable for the isolation of VRL-2 cDNA.

Any of a variety of procedures may be used to clone VRL-2 cDNA. These methods include, but are not limited to, direct functional expression of the VRL-2 cDNA following the construction of an VRL-2-containing cDNA library in an appropriate expression vector system. Another method is to screen an VRL-2-containing cDNA library constructed in a bacteriophage or plasmid shuttle vector with a labelled oligonucleotide probe designed from the amino acid sequence of the VRL-2 protein. The preferred method includes screening an VRL-2-containing cDNA library constructed in a bacteriophage or plasmid shuttle vector with a partial cDNA encoding the VRL-2 protein. This partial cDNA is obtained by the specific PCR amplification of VRL-2 DNA fragments through the design of degenerate oligonucleotide primers from the amino acid sequence known for other capsaicin receptors.

It is readily apparent to those skilled in the art that other types of libraries, as well as libraries constructed from other cells or cell types, may be useful for isolating VRL-2-encoding DNA. Other types of libraries include, but are not limited to, cDNA libraries derived from other cells or cell lines other than human erythroleukemia cells, and genomic DNA libraries.

It is readily apparent to those skilled in the art that suitable cDNA libraries may be prepared from cells or cell lines which have VRL-2 activity. The selection of cells or cell lines for use in preparing a cDNA library to isolate VRL-2 cDNA may be done by first measuring cell associated VRL-2 activity using the known labelled ligand binding assay cited above and used herein.

Preparation of cDNA libraries can be performed by standard techniques well known in the art. Well known cDNA library construction techniques can be found for example, in Maniatis, T., Fritsch, E. F., Sambrook, J., Molecular Cloning: A Laboratory Manual (Cold Spring Harbor Laboratory, Cold Spring Harbor, N.Y., 1982).

It is also readily apparent to those skilled in the art that DNA encoding VRL-2 may also be isolated from a suitable genomic DNA library. Construction of genomic DNA libraries can be performed by standard techniques well known in the art. Well known genomic DNA library construction techniques can be found in Maniatis, T., Fritsch, E. F., Sambrook, J. in Molecular Cloning: A Laboratory Manual (Cold Spring Harbor Laboratory, Cold Spring Harbor, N.Y., 1982).

In order to clone the VRL-2 gene by one of the preferred methods, the amino acid sequence or DNA sequence of VRL-2 or a homologous protein is necessary. To accomplish this, VRL-2 protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial VRL-2 DNA fragment.

Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the VRL-2 sequence but others in the set will be capable of hybridizing to VRL-2 DNA even in the presence of DNA oligonucleotides with mismatches. The mismatched DNA oligonucleotides may still sufficiently hybridize to the VRL-2 DNA to permit identification and isolation of VRL-2 encoding DNA.

Using one of the preferred methods, cDNA clones encoding VRL-2 polypeptide are isolated in a two-stage approach employing polymerase chain reaction (PCR) based technology and cDNA library screening. In the first stage, NH.sub.2-terminal and internal amino acid sequence information from the purified VRL-2 or a homologous protein is used to, design degenerate oligonucleotide primers for the amplification of VRL-2-specific DNA fragments. In the second stage, these fragments are cloned to serve as probes for the isolation of full length cDNA from a cDNA library derived from human erythroleukemia cells.

The polynucleotides of the present invention may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding paralogs from human sources and orthologs and paralogs from species other than human) that have a high sequence similarity to SEQ ID NO:1 or SEQ ID NO:3.

The probes or primers of the present invention generally comprise at least 15 nucleotides, preferably, at least 30 nucleotides and may have at least 50 nucleotides. Particularly preferred probes will have between 30 and 50 nucleotides. Particularly preferred primers will have between 20 and 25 nucleotides.

A polynucleotide encoding a polypeptide of the present invention, including homologs from species other than human, may be obtained by a process which comprises the steps of screening an appropriate library under stringent hybridization conditions with a labeled probe having the sequence of SEQ ID NO:1 or SEQ ID NO:3 or a fragment thereof and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Thus the present invention also includes polynucleotides obtainable by screening an appropriate library under stringent hybridization conditions with a labeled probe having the sequence of SEQ ID NO:1 or SEQ ID NO:3, or a fragment thereof.

The skilled artisan will appreciate that, in many cases, an isolated cDNA sequence will be incomplete, in that the region coding for the polypeptide is short at the 5′ end of the cDNA. This is a consequence of reverse transcriptase, an enzyme with inherently low ‘processivity’ (a measure of the ability of the enzyme to remain attached to the template during the polymerization reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example. Frohman et al., PNAS USA 85, 8998-9002, 1988). Recent modifications of the technique, exemplified by the Marathon™ technology (Clontech Laboratories Inc.) for example, have significantly simplified the search for longer cDNAs.

Expression Vectors

Recombinant VRL-2 polypeptides of the present invention may be prepared by processes well known in the art from genetically engineered host cells comprising expression vectors. Accordingly, the present invention relates to an expression vector for the expression of a human vanilloid receptor-ike protein in a recombinant host cell wherein said expression vector contains the above-identified DNA molecule.

The cloned VRL-2 cDNA obtained through the methods described above may be recombinantly expressed by molecular cloning into an expression vector containing a suitable promoter and other appropriate transcription regulatory elements, and transferred into prokaryotic or eukaryotic host cells to produce recombinant VRL-2 polypeptides. Techniques for such manipulations can be found described in Maniatis, T, et al., supra, and are well known in the art.

Expression vectors are defined herein as DNA sequences that are required for the transcription of cloned DNA and the translation of their mRNAs in an appropriate host. Such vectors can be used to express eukaryotic DNA in a variety of hosts such as bacteria, bluegreen algae, plant cells, insect cells and animal cells.

Specifically designed vectors allow the shuttling of DNA between hosts such as bacteria-yeast or bacteria-animal cells. An appropriately constructed expression vector should contain an origin of replication for autonomous replication in host cells, selectable markers, a limited number of useful restriction enzyme sites, a potential for high copy number, and active, promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

A variety of mammalian expression vectors may be used to express recombinant VRL-2 in mammalian cells. Commercially available mammalian expression vectors which may be suitable for recombinant VRL-2 expression, include but are not limited to, pMClneo (Stratagene), pXT1 (Stratagene), pSG5 (Stratagene), pcDNA1, pcDNA3.1 (Invitrogen), EBO-pSV2-neo (ATCC 37593) pBPV-1 (8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

Host Cells Containing Expression Vector

DNA encoding the VRL-2 polypeptide may also be cloned into an expression vector for expression in a host cell. Host cells may be prokaryotic or eukaryotic, including but not limited to bacteria, fungal, insect, animal (including mammalian) and plant cells. Suitable bacterial cells inlcude Streptococci, Staphylococci, E. coli, Streptomyces and Bacillus subtilis cells. Suitable fungal cells include yeast cells and Aspergillus cells. The animal cells include cell lines of human, bovine, porcine, monkey and rodent origin. Suitable insect cells include Drosophila S2 and Spodoptera Sf9 cells. Suitable animal cells include CHO, COS, HeLa, C127, 3T3, BHK, HEK 293 and Bowes melanoma cells. Cell lines derived from mammalian species which may be suitable and which are commercially available, include CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

The expression vector may be introduced into host cells via any one of a number of techniques including but not limited to transformation, transfection, protoplast fusion, and electroporation. The expression vector-containing cells are individually analyzed to determine whether they produce VRL-2 protein. Identification of VRL-2 expressing cells may be done by several means, including but not limited to immunological reactivity with anti-VRL-2 antibodies, and the presence of host cell-associated VRL-2 activity.

Expression of VRL-2 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

To determine the VRL-2 cDNA sequence(s) that yields optimal levels of receptor activity and/or VRL-2, VRL-2 cDNA molecules including but not limited to the following can be constructed: the full-length open reading frame of the VRL-2 cDNA and various constructs containing portions of the cDNA encoding only specific domains of the receptor protein or rearranged domains of the protein. All constructs can be designed to contain none, all or portions of the 5′ and/or 3′ untranslated region of VRL-2 cDNA. VRL-2 activity and levels of protein expression can be determined following the introduction, both singly and in combination, of these constructs into appropriate host cells. Following determination of the VRL-2 cDNA cassette yielding optimal expression in transient assays, this VRL-2 cDNA construct is transferred to a variety of expression vectors (including recombinant viruses), including but not limited to those for mammalian cells, plant cells, insect cells, oocytes, E. Coli, and yeast cells.

Mammalian cell transfectants are assayed for both the levels of VRL-2 receptor activity and levels of VRL-2 protein by the following methods. Assessing VRL-2 receptor activity involves the direct introduction of a labelled ligand to the cells and determining the amount of specific binding of the ligand to the VRL-2-expressing cells. Binding assays for receptor activity are known in the art (Frey et al., 1993, Eur. J. Pharmacol., 244, pp 239-250).

Levels of VRL-2 protein in host cells is quantitated by a variety of techniques including, but not limited to, immunoaffinity and/or ligand affinity techniques. VRL-2-specific affinity beads or VRL-2-specific antibodies are used to isolate .sup.35 S-methionine labelled or unlabelled VRL-2 protein. Labelled VRL-2 protein is analyzed by SDS-PAGE. Unlabelled VRL-2 protein is detected by Western blotting, ELISA or RIA assays employing VRL-2 specific antibodies.

Following expression of VRL-2 in a host cell, VRL-2- protein may be recovered to provide VRL-2 in active form, capable of binding VRL-2-specific ligands. Several VRL-2 purification procedures are available and suitable for use.

Recombinant VRL-2 may be purified from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography.

Thus, the present invention also provides a process for producing the VRL-2 receptor polypeptides, which comprises culturing a host cell comprising the expression vector containing the above DNA molecule under conditions sufficient for the production of said polypeptide and recovering the polypeptide from the culture medium. The conditions for producing the polypeptides can be easily designed by a skilled person. Well known techniques for refolding proteins may be employed to regenerate active conformation when the polypeptide is denatured during intracellular synthesis, isolation and or purification.

Antibodies

The present invention further provides antibodies immunospecific for the VRL-2 receptor polypeptide.

The antibodies may be obtained by administering the polypeptides or epitope-bearing fragments, analogs or cells to an animal, preferably a non-human animal, according to known techniques. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozboret al., Immunology Today (1983) 4:72) and the EBV-hybridoma technique (Cole et al., Monoclonal Antibodies and Cancer Therapy 77-96, Alan R. Liss, Inc., 1985). Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies. The above-described antibodies may be employed to isolate or to identify clones expressing the polypeptide or to purify the polypeptides by affinity chromatography. Antibodies against polypeptides of the present invention may also be employed to treat the diseases, amongst others.

Diagnostic Tool

The DNA molecules of the present invention can be used as diagnostic reagents. Detection of a mutated form of the gene characterized by the polynucleotide of this invention, which is associated with a dysfunction, will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids for diagnosis may be obtained from a subject's cells, such as from blood, urine, saliva tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled VRL-2 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (see, e.g., Myers et al. Science (1985) 230: 1242).

Sequence changes at specific locations may also be revealed by nuclease protection assays, such as RNase and S I protection or the chemical cleavage method (see Cottonet al., Proc Nati Acad Sci USA (1985) 85: 4397-440 1). In another embodiment, an array of oligonucleotides probes comprising VRL-2 nucleotide sequence or fragments thereof can be constructed to conduct efficient screening of e,g., genetic mutations. Array technology methods are well known and have general applicability and can be used to address a variety of questions in molecular genetics including gene expression, genetic linkage, and genetic variability (see for example: M. Chee et al. Science, Vol 274, pp 610-613 (1996)).

The diagnostic assays offer a process for diagnosing or determining a susceptibility to the VRL-2 related diseases through detection of mutation in the VRL-2 gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR. RNase protection, Northern plotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

In another aspect, the present invention relates to a diagnostic-kit which comprises the DNA molecule of the present invention or an antibody to the polypeptide of the present invention. If desired, the diagnostic kit can comprise other materials which can be used in known diagnostic kits. Such a kit can be used in diagnosing a disease or suspectability to a disease, particularly pain, chronic pain, neuropathic pain, postoperative pain, rheumatoid arthritic pain, neuralgia, neuropathies, algesia, nerve injury, ischaemia, neurodegeneration, stroke, incontinence and inflammatory disorders, amongst others.

Screening Methods

The novel VRL-2 receptor of the present invention is suitable for use in an assay procedure for identification of modulators which modulate the function of the VRL-2 receptor activity. Thus, this invention also provides a method for screening modulators which modulate the function of the VRL-2 polypeptides. As used herein, “modulating function of a receptor” or “modulating receptor activity” includes the inhibition or activation of the receptor, and also includes directly or indirectly affecting the normal regulation of the receptor activity. As used herein, the term “modulator” means compounds, agonists, antagonists, ligands, substrates and enzymes which directly or indirectly affect regulation of the receptor activity.

The VRL-2 receptor of the present invention may be obtained from both native and recombinant sources for use in an assay procedure to identify receptor modulators. In general, an, assay procedure to identify VRL-2 receptor modulators may comprise contacting the VRL-2 polypeptide (or the cells or membranes bearing the polypeptide) of the present invention with a test compound or sample which expects to contain a VRL-2 receptor modulator, and detecting an activity to modulate the function of the VRL-2 receptor (e.g., binding activity). The test compounds or samples may be tested directly on, for example, purified receptor, subcellular fractions of receptor-producing cells, and/or whole cells expressing the receptor. The test compound or sample may be added to the receptor in the presence or absence of a known labelled or unlabelled receptor ligand. Further, these screening methods may test whether the candidate compound results in a signal generated by activation or inhibition of the polypeptide, using detection systems appropriate to the cells bearing the polypeptide. The modulating activity of the test compound or sample may be determined by, for example, analyzing the ability of the test compound or sample to bind to the receptor, activate the receptor, inhibit receptor activity, inhibit or enhance the binding of other compounds to the receptor, modifying receptor regulation, or modifying an intracellular activity.

The other known screening methods can be used in this invention. Such known methods can be found in PCT International Publication No. WO 99/37765, Page 13, Line 5 to Page 14, Line 31. In addition, the screening method of this invention covers a counter-assay to identify a certain compound which does not have a VRL-2 modulating activity.

The above-mentioned screening method can be practiced by use of a screening kit for identifying modulators for polypeptides of the present invention, which comprises (a) a polypeptide of the present invention; (b) a recombinant cell expressing a polypeptide of the present invention; or (c) a cell membrane expressing a polypeptide of the present invention. Any known materials or compoents which can be used for a screening kit can be added to the screening kit of this invention.

The modulators of VRL-2 receptor activity are useful in treating disease states involving the VRL-2 receptor activity as mentioned above.

Pharmaceutical Composition

The present invention further provides a pharmaceutical composition for treatment of conditions associated with biological function of a human vanilloid receptor-like protein, which comprises a therapeutically effective amount of a modulator (e.g., agonist or antagonist) obtained by the above-mentioned screening methods and a pharmaceutically acceptable carrier. The conditions associated with biological function of the VRL-2 receptor include pain, nociceptive pain, chronic pain, neuropathic pain, postoperative pain, cancer pain, rheumatoid arthritic pain, osteoarthritis, diabetic neuropathies, neuralgia, neuropathies, algesia, nerve injury, muscle-skeletal pain, low back pain, neurodegeneration, stroke, inflammatory disorders, athma, allergy, urogenital disorders, incontinence, hypertension, hypotension, perivasular disease and the like. Thus, the pharmaceutical compositions of this invention are useful as drugs for treating such diseases.

The pharmaceutical composition of this invention can be administered via either the oral, parenteral or topical routes to mammals. In general, these compounds are most desirably administered to humans in doses ranging from 0.1 mg to 750 mg per day, preferably from 10 mg to 500 mg per day, although variations will necessarily occur depending upon the weight and condition of the subject being treated, the disease state being treated and the particular route of administration chosen.

The compounds of the present invention may be administered alone or in combination with pharmaceutically acceptable carriers or diluents by either of the above routes previously indicated, and such administration can be carried out in single or multiple doses. More particularly, the novel therapeutic agents of the invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various nontoxic organic solvents, etc. Moreover, oralpharmaceutical compositions can be suitably sweetened and/or flavored. In general, the therapeutically-effective compounds of this invention are present in such dosage forms at concentration levels ranging 5% to 70% by weight, preferably 10% to 50% by weight.

For oral administration, tablets containing various excipients such as microcrystalline cellulose, sodium citrate, calcium carbonate, dipotassium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatine capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH>8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well-known to those skilled in the art. Additionally, it is also possible to administer the compounds of the present invention topically when treating inflammatory conditions of the skin and this may preferably be done by way of creams, jellies, gels, pastes, ointments and the like, in accordance with standard pharmaceutical practice.

Transgenic Animals

The polypeptides and/or DNA molecules of the present invention can be used to generate genetically modified non-human animals or site specific gene modifications in cell lines. The term “transgenic” is intended to encompass genetically modified animals having, for example, a deletion or other knock-out of VRL-2 receptor gene activity, an exogenous VRL-2 receptor gene that is stably transmitted in the host cells, a “knock-in” having altered VRL-2 receptor gene expression, or an exogenous VRL-2 receptor polypeptide promoter operably linked to a reporter gene. Homozygous and heterozygous knock-outs and knock-ins of VRL-2 receptor polypeptide function are particularly preferred.

Transgenic animals can be made according to methods known to skilled persons in the art. For example, the transgenic animals may be made through homologous recombination, where the endogenous VRL-2 receptor locus is altered. Alternatively, a nucleic acid construct is randomly integrated into the genome. Vectors for stable integration include plasmids, retroviruses and other animal viruses, YACs, and the like. Of interest are transgenic mammals, preferably a mammal from a genus selected from the group consisting of Mus (e.g., mice), Rattus (e.g., rats), Oryctologus (e.g., rabbits) and Mesocricetus (e.g., hamsters). More specific descriptions about preparation and use of the transgenic animals can be found in WO 99/37675, Page 34, Line 36 to Page 37, Line 12.

### EXAMPLES

The following examples are provided for the purpose of illustrating the present invention without, however, limiting the same thereto.

### Example 1

### Cloning of hVRL-2 cDNA

A BLAST search against LifeSeq™ database (Incyte) was made, using the polynucleotide and polypeptide sequences of the rat vanilloid receptor subtype 1 (hereinafter referred to as “VR1”). Six cDNA clones were obtained that contained nucleotide sequences highly homologous to the rat VR1. Five clones out of six were human homologs of rat VR1 or human vanilloid receptor-like protein 1 (hereinafter referred to as “VRL-1”). However, one cDNA clone did not match either of VR1 or VRL-1, and encoded a novel human receptor homologous to vanilloid receptor subtypes. This cDNA was designated as “VRL-2”. This cDNA clone did not contain the entire open reading frame.

Further, a full-length cDNA cloning of human VRL-2 was performed. To identify start codon of VRL-2, two primers (5′-GCT GTC GGG GAA GAG GCG GGC ACA CTT G-3′ (SEQ ID NO:5) and 5′-GCA GCA GTT CAT TGA TGG GCT CCA CAG C (SEQ ID NO:6)) designed from the sequence data of incyte clone and human pancreas Marathon-Ready™ cDNA (Clontech, Palo Alto, Calif.) were used. 5′ rapid amplification of cDNA ends (5′RACE) was performed under the following conditions: 94° C. for 1 min and 68° C. for 2 min, 30 cycles. The amplified 1.1 Kb RACE product were cloned and sequenced by automated DNA sequencer ABI PRISM 310 (PE Biosystems, Foster City, Calif.) produced by TOYOBO. These sequence data showed that 5′RACE, products contained two kinds of 5′ portion that possess a potential initiator codon downstream of an in-frame termination codon. VRL-2 constituted a subfamily that contained at least two spliced variants. One clone was designated as VRL-2a, and the other was designated as VRL-2b.

To isolate complete open reading flame of VRL2a and VRL-2b, PCR was performed using Advantage-HF PCR Kit (Clontech, Palo Alto, Calif.) and human kidney cDNA library (Clontech, Palo Alto, Calif.). Two sets of primers were designed from sequence data of 5′RACE products and Incyte clone; VRL-2a Forward 5′-ATC TGC GCA TGA AGTTCC AG-3′ (SEQ ID NO:7), VRL-2b Forward 5′-GAC ATC GCG GAG CGC ACC GGC AAC ATG-3′ (SEQ ID NO:8) and VRL-2a,b Reverse 5′-GCT GGA CTA GAA ATG AGT GGG CAG AGA A (SEQ ID NO:9). PCR was performed under the following conditions: 94° C. for 20 sec, 58° C. for 30 sec and 72° C. for 30 sec, 25 cycles. PCR products of 2.6 kb (VRL-2a) and 1.9 kb (VRL-2b) were cloned and sequenced by automated DNA sequencer ABI PRISM 310 (PE Biosystems, Foster City, Calif.; produced by TOYOBO Gene Analysis. The amino acid sequences of VRL-2a and VRL-2b are shown in SEQ ID NO:2 and SEQ ID NO:4, respectively.

### Example 2

### Expression of hVRL-2 in Mammalian Cells

A cDNA encoding human VRL2a was subcloned into the eukaryotic expression vector pcDNA3.1 (Invitrogen). CHO-K1 cells and HEK 293 cells were plated onto poly-l-lysine coated glass-bottom dishes and transiently transfected with expression vector and pIRES2-EGFP (Clonetech) (total 1.0 μg) and 3 μl FuGENE6 transfection reagent (Roche) in 97 μl of OptiMEM medium (Life Technologies) per 35 mm dish.

### Example 3

### Functional Analysis of hVRL-2

Activities of VRL-2 as a cation channel are readily detected with fluorescent dyes and conventional microscopy. The VRL-2-expressing cells (as obtained in Example 2) plated on glass coverslips are loaded with calcium indicators such as fura-2 or fluo-3, or a membrane potential indicator DiBAC4 in Hank's balanced salt buffer. The osmotic, mechanical or thermal stimuli are applied to the VRL-2-expressing cells loaded with the fluorescent dyes, and changes in fluorescence are monitored by a single cell imaging system mounted on an inverted fluorescence microscopy.

In this example, Ca²⁺ measurements were carried out for 24 to 48 hours after transfection. Ca⁺ measurements in single cells were made using the fluorescent Ca²⁺ indicator fura-2 in combination with a fluorescence imaging system MERLIN (OLYMPUS) mounted on an inverted fluorescence microscopy (IX70, OLYMPUS). For Ca²⁺ imaging, cells were loaded with 5 μM fura-2 acetoxymethylester (Dojin) for 30 min at room temperature in isotonic (300 mosmol I⁻¹) measuring buffer containing 88 mM NaCl, 5 mM KCl, 1 mM MgCl₂, 1 mM CaCl₂, 5.5 mM glucose and 10 mM HEPES at pH7.4. The osmolarity was adjusted by addition of appropriate amounts of mannitol. A sequence of fluorescent images (340 nm/380 nm excitation) was taken to monitor Ca²⁺ influx response in cells with or without expression of GFP before and after the osmotic change. Cells were initially kept in isotonic solution. Hypotonic challenge was achieved by perfusion with 200 mosmol I⁻¹ measuring buffer. The solution of lower osmolarity was obtained by reducing the mannitol concentration. The effective assay buffer volume was 2 ml, and perfusion was carried out at room temperature at a flow rate of 3 ml min⁻¹. Complete exchange of the perfusion solution was assumed after 2 min. Non-transfected cells were used as controls. Other control experiments were carried out using cells transfected with pcDNA3.1.

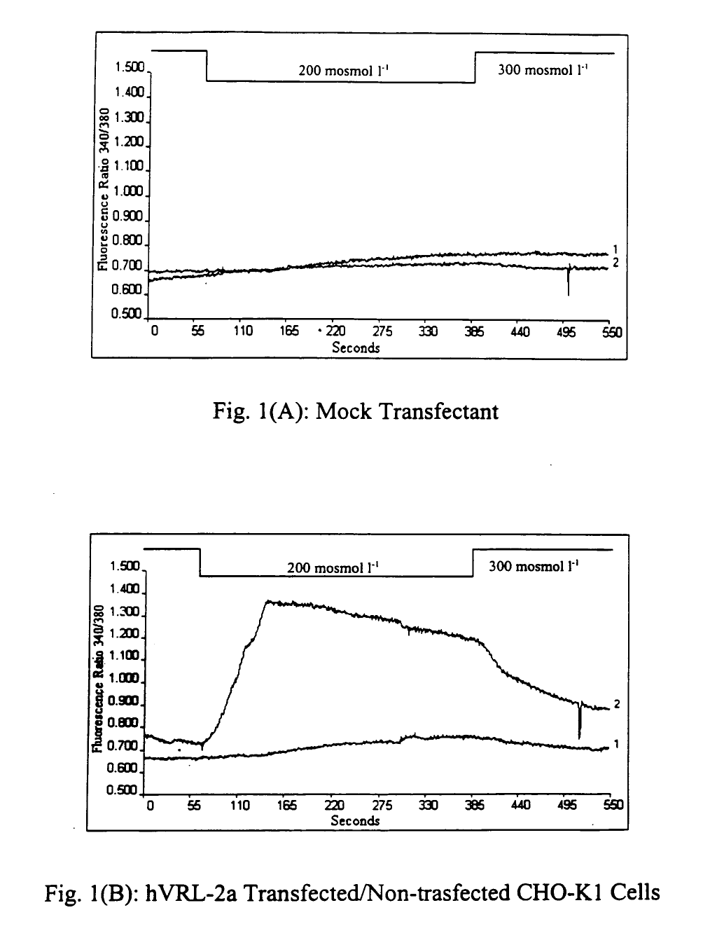

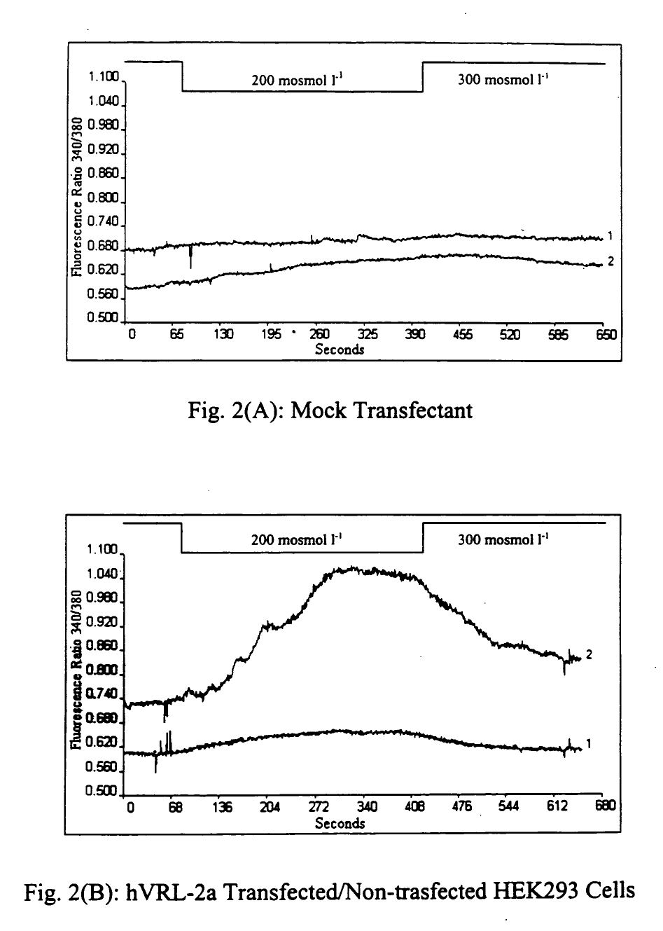

The results of the experiments can be seen in FIGS. 1(A), 1(B), 2(A) and 2(B).

FIGS. 1(A) and 1(B) show Ca²⁺ increase in hVRL-2 transfected CHO-K1 cells in response to a reduction in extracellular osmolarity. In FIG. 1(A), Lines 1 and 2 show response curves of cells without expression vectors (Mock transfection). In FIG. 1(B), Line 1 shows a response curve of a GFP-negative cell (non-transfected cell), and Line 2 shows a response curve of a GFP-positive cell (hVRL-2a transfected cell).

FIGS. 2(A) and 2(B) show Ca²⁺ increase in hVRL-2 transfected HEK293 cells in response to a reduction in extracellular osmolarity. In FIG. 2(A), Lines 1 and 2 show response curves of cells without expression vectors (Mock transfection). In FIG. 2(B), Line 1 shows a response curve of a GFP-negative cell (non-transfected cell), and Line 2 shows a response curve of a GFP-positive cell (hVRL-2a transfected cell).

As can be seen in these Figures, upon reduction of extracellular osmolarity, intracellular Ca²⁺ increased significantly in the hVRL2a transfected cells, but not in control cells. This response was reversible, and increasing the osmolarity led to a decrease in intracellular Ca²⁺.

All publications, including but not limited to patents and patent applications, cited in this specification are herein incorporated by reference as if each individual publication were specifically and individually indicated to be incorporated by reference herein as though fully set forth. In particular, the definition of technical terms described in WO 99/37765 can be used as reference.

1. A polypeptide selected from:
(a) a polypeptide comprising an amino acid sequence of SEQ ID NO:2 or SEQ ID NO:4, or variants thereof; and
(b) a polypeptide having the deduced amino acid sequence translated from the polynucleotide sequence SEQ ID NO:1 or SEQ ID NO:3, or variants thereof.

2. A polypeptide of claim 1, comprising an amino acid sequence that has at least 75% identity to the an amino acid sequence of SEQ ID NO:2 or SEQ ID NO:4.

3. A polypeptide of claim, comprising an amino acid sequence that has at least 85% identity to the an amino acid sequence of SEQ ID NO:2 or SEQ ID NO:4.

4. A polypeptide of claim 1, comprising an amino acid sequence that has at least 95% identity to the an amino acid sequence of SEQ ID NO:2 or SEQ ID NO:4.

5. An isolated and/or purified polynucleotide selected from:
(a) a polynucleotide encoding the polypeptide comprising an amino acid sequence of SEQ ID NO:2 or SEQ ID NO:4;
(b) a polynucleotide having a nucleotide sequence of the 85th to 2700th nucleotides in the nucleotide sequence of SEQ ID NO:1, or a nucleotide sequence of the 43rd to 1851st nucleotides in the nucleotide sequence of SEQ ID NO:3 or variants thereof;
(c) a polynucleotide comprising a nucleotide sequence that has at least 70% identity to the polynucleotide of (a) or (b);
(d) a complement to the polynucleotide of any one of (a) to (c);
(e) a polynucleotide comprising a nucleotide sequence which is capable of hybridising to the polynucleotide of any one of (a) to (d); and
(f) a polynucleotide fragment of the polynucleotide of any one of (a) to (e).

6. A polynucleotide of claim 5, comprising a nucleotide sequence that has at least 75% identity to the nucleotide sequence of the polynucleotide (b).

7. A polynucleotide of claim 5, comprising a nucleotide sequence that has at least 85% identity to the nucleotide sequence of the polynucleotide (b).

8. A polynucleotide of claim 5, comprising a nucleotide sequence that has at least 95% identity to the nucleotide sequence of the polynucleotide (b).

9. A polynucleotide probe or primer comprising at least 15 contiguous nucleotides of the polynucleotide of any one of claim 5.

10. An expression vector for the expression of a human vanilloid receptor-ike protein in a recombinant host cell wherein said expression vector contains the polynucleotide of claim 5.

11. A host cell which expresses a human vanilloid receptor-like protein wherein said host cell contains the expression vector of claim 10.

12. A process for producing a polypeptide of claim 1, which comprises culturing a host cell of claim 11 under conditions sufficient for the production of said polypeptide and recovering the polypeptide from the culture medium.

13. An antibody immunospecific for a polypeptide of claim 1.

14. A diagonostic kit for diagnosing a disease or a susceptibility to a disease associated with biological function of a human vanilloid receptor-like protein, which comprises a polynucleotide of claim 5, or an antibody to a polypeptide of claim 1.

15. A method for screening to identify modulators which modulate function of a polypeptide of claim 1, which comprises contacting a test compound or sample with the polypeptide, and detecting an activity of the test compound or sample to modulate the function of the polypeptide.

16. A modulator obtainable by a screening method of claim 15.

17. A pharmaceutical composition for treatment of conditions associated with biological function of a human vanilloid receptor-like protein, which comprises a therapeutically effective amount of a modulator of claim 16 and a pharmaceutically acceptable carrier.

18. A non-human transgenic animal model for vanilloid receptor-like gene function wherein the transgenic animal has an alteration in vanilloid receptor-like function relative to a normal animal of the same species.


SEQUENCE LISTING (9 sequences)

SEQ ID NO: 1 - DNA, 2749 bp, Human
aaccgcttgaggatttaagctttgccactttggctccggagcaagggcagagggctgagcagtgcagacgggcctggggcaggcatggcggattccagcgaaggcccccgcgcggggcccggggaggtggctgagctccccggggatgagagtggcaccccaggtggggaggcttttcctctctcctccctggccaatctgtttgagggggaggatggctccctttcgccctcaccggctgatgccagtcgccctgctggcccaggcgatgggcgaccaaatctgcgcatgaagttccagggcgccttccgcaagggggtgcccaaccccatcgatctgctggagtccaccctatatgagtcctcggtggtgcctgggcccaagaaagcacccatggactcactgtttgactacggcacctatcgtcaccactccagtgacaacaagaggtggaggaagaagatcatagagaagcagccgcagagccccaaagcccctgcccctcagccgccccccatcctcaaagtcttcaaccggcctatcctctttgacatcgtgtcccggggctccactgctgacctggacgggctgctcccattcttgctgacccacaagaaacgcctaactgatgaggagtttcgagagccatctacggggaagacctgcctgcccaaggccttgctgaacctgagcaatggccgcaacgacaccatccctgtgctgctggacatcgcggagcgcaccggcaacatgcgggagttcattaactcgcccttccgtgacatctactatcgaggtcagacagccctgcacatcgccattgagcgtcgctgcaaacactacgtggaacttctcgtggcccagggagctgatgtccacgcccaggcccgtgggcgcttcttccagcccaaggatgaggggggctacttctactttggggagctgcccctgtcgctggctgcctgcaccaaccagccccacattgtcaactacctaacggagaacccccacaagaaggcggacatgcggcgccaggactcgcgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggatctttcagcacatcatccggcgggaggtgacggatgaggacacacggcacctgtcccgcaagttcaaggactgggcctatgggccagtgtattcctcgctttatgacctctcctccctggacacgtgtggggaagaggcctccgtgctggagatcctggtgtacaacagcaagattgagaaccgccacgagatgctggctgtggagcccatcaatgaactgctgcgggacaagtggcgcaagttcggggccgtctccttctacatcaacgtggtctcctacctgtgtgccatggtcatcttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctccctcctgaacccgtgtgccaacatgaaggtgtgcaatgaggaccagaccaactgcacagtgcccacttacccctcgtgccgtgacagcgagaccttcagcaccttcctcctggacctgtttaagctgaccattggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaacaagaactcgaacccggacgaggtggtggtgcctctggacagcatggggaacccccgctgcgatggccaccagcagggttacccccgcaagtggaggactgatgacgccccgctctagggactgcagcccagccccagcttctctgcccactcatttctagtccagc

SEQ ID NO: 2 - protein, 870 aa, Human
MADSSEGPRAGPGEVAELPGDESGTPGGEAFPLSSLANLFEGEDGSLSPSPADASRPAGPGDGRPNLRMKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYSSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTDDAP

SEQ ID NO: 3 - DNA, 1900 bp, Human
gcggccgctgaattctaggacatcgcggagcgcaccggcaacatgcgggagttcattaactcgcccttccgtgacatctactatcgaggggagctgcccctgtcgctggctgcctgcaccaaccagccccacattgtcaactacctgacggagaacccccacaagaaggcggacatgcggcgccaggactcgcgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggatctttcagcacatcatccggcgggaggtgacggatgaggacacacggcacctgtcccgcaagttcaaggactgggcctatgggccagtgtattcctcgctttatgacctctcctccctggacacgtgtggggaagaggcctccgtgctggagatcctggtgtacaacagcaagattgagaaccgccacgagatgctggctgtggagcccatcaatgaactgctgcgggacaagtggcgcaagttcggggccgtctccttctacatcaacgtggtctcctacctgtgtgccatggtcatcttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctccctcctgaacccgtgtgccaacatgaaggtgtgcaatgaggaccagaccaactgcacagtgcccacttacccctcgtgccgtgacagcgagaccttcagcaccttcctcctggacctgtttaagctgaccatcggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaacaagaactcgaacccggacgaggtggtggtgcctctggacagcatggggaacccccgctgcgatggccaccagcagggttacccccgcaagtggaggactgatgacgccccgctctagggactgcagcccagccccagcttctctgcccactcatttctagtccagc

SEQ ID NO: 4 - protein, 602 aa, Human
MREFINSPFRDIYYRGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYSSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTDDAPL

SEQ ID NO: 5 - DNA, 28 bp, Homo sapiens
gctgtcggggaagaggcgggcacacttg

SEQ ID NO: 6 - DNA, 28 bp, Homo sapiens
gcagcagttcattgatgggctccacagc

SEQ ID NO: 7 - DNA, 20 bp, Homo sapiens
atctgcgcatgaagttccag

SEQ ID NO: 8 - DNA, 27 bp, Homo sapiens
gacatcgcggagcgcaccggcaacatg

SEQ ID NO: 9 - DNA, 28 bp, Homo sapiens
gctggactagaaatgagtgggcagagaa